(19) United States
(12) Plant Patent Application Publication
(10) Pub. No.: US 2017/0172030 P1
(43) Pub. Date: Jun. 15, 2017

(54) Interspecific plum hybrid rootstock named 'Diprec'

(50) ;

(71) Applicant: Michael Neumüller, Hallbergmoos (DE)

(72) Inventor: Michael Neumüller, Hallbergmoos (DE)

(21) Appl. No.: 14/757,289

(22) Filed: Dec. 10, 2015

(51) Int. Cl. A01H 5/08

(52) U.S. Cl. PLT/184

(57) ABSTRACT

A new interspecific plum hybrid rootstock named ‘Diprec’ is disclosed. ‘Diprec’ exhibits a resistance to Plum Pox Virus (PPV) mediated by a hypersensitive response to PPV. ‘Diprec’ is used as a rootstock for commercial fruit trees.

### CROSS-REFERENCE TO RELATED APPLICATIONS

None

### STATEMENT REGARDING FEDERALLY SPONSORED RESEARCH OR DEVELOPMENT

None

Genus and species: Prunus domestica×Prunus cerasifera.

Variety denomination: ‘DIPREC’.

### BACKGROUND OF THE INVENTION

‘Diprec’ is a product of a controlled breeding program carried out by the inventor in Hallbergmoos, Germany. ‘Diprec’ is an interspecific plum hybrid rootstock that resulted from a cross between Prunus domestica and Prunus cerasifera. Throughout several generations of asexual propagation, ‘Diprec’ has been observed to retain its distinctive characteristics and remain true to type. ‘Diprec’ exhibits a resistance to Plum Pox Virus (PPV) mediated by a hypersensitive response to PPV.

### BRIEF DESCRIPTION OF THE PHOTOGRAPH

FIG. 1 illustrates an immature ‘Diprec’ rootstock plant.

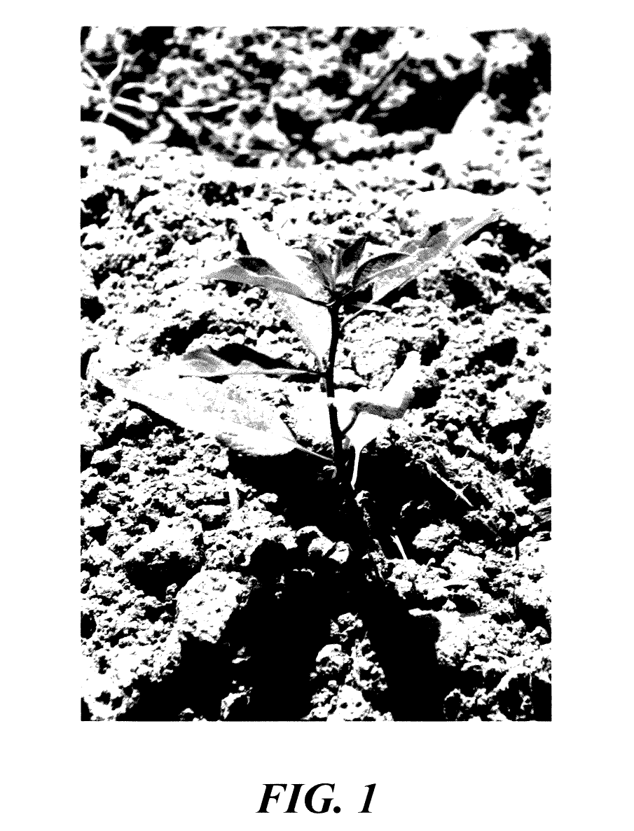

FIG. 2 illustrates flowers of a mature ‘Diprec’ rootstock plant.

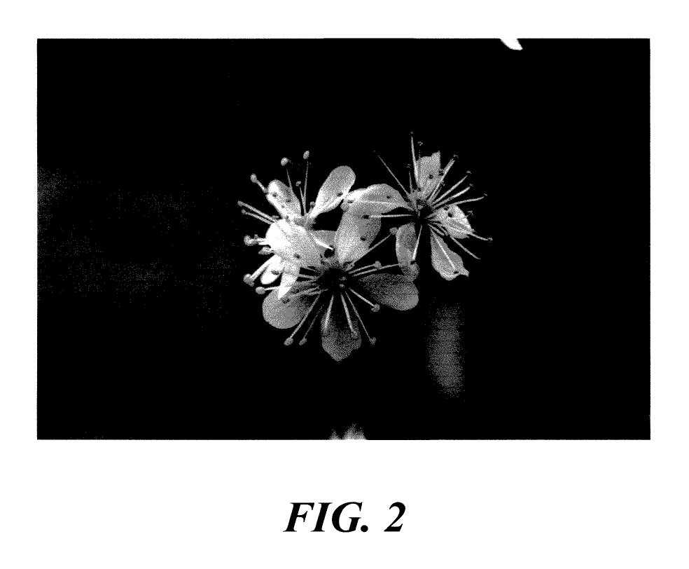

FIG. 3 illustrates fruit on a mature ‘Diprec’ rootstock plant.

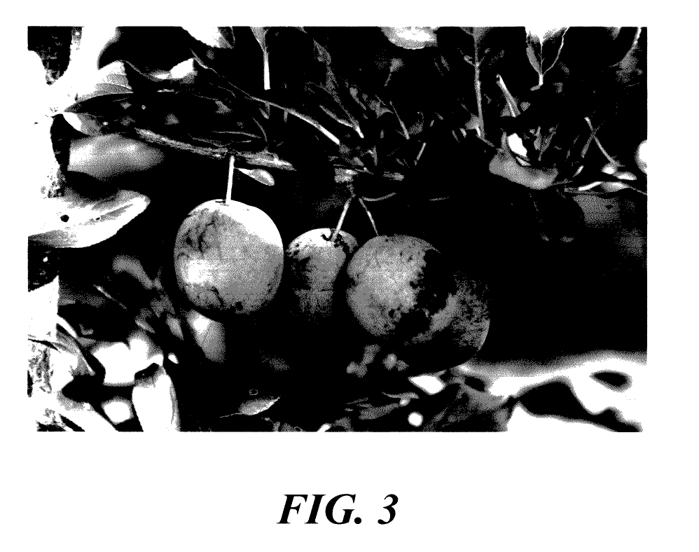

FIG. 4 illustrates fruit produce from a mature ‘Diprec’ plant.

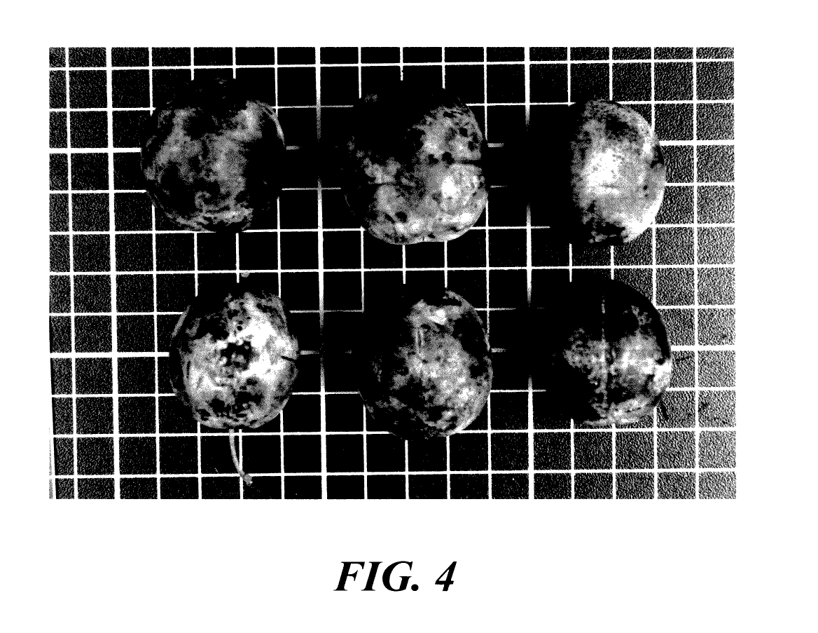

The colors of these illustrations may vary with lighting conditions and, therefore, color characteristics of this new variety should be determined with reference to the observations described herein, rather than from these illustrations alone.

### DETAILED BOTANICAL DESCRIPTION

The following description is based on observations made in Germany. It should be understood that the characteristics described will vary somewhat depending upon cultural practices and climatic conditions, and can vary with location and season. Quantified measurements are expressed as an average of measurements taken from a number of individual plants of the new variety. The measurements of any individual plant or any group of plants, of the new variety may vary from the stated average. All color references are based on The Royal Horticultural Society Color Chart.
Use: Rootstock for commercial fruit trees.
Disease susceptibility: Resistance to Plum Pox Virus (PPV) mediated by hypersensitivity to PPV.
Suckering: Low suckering.
Vigor: European plum trees grafted onto ‘Diprec’ rootstock show medium vigour, similar to ‘Ishtara’ or ‘St. Julien A.’
Flowers: Early flowering.
Fertility: Highly fertile.
Fruit: Fruits ripen in late August in Germany.

1. What is claimed is a new and distinct interspecific plum hybrid rootstock as shown and described herein.
